SEQUENCE LISTING (29 sequences)

SEQ ID NO: 1 - protein, 856 aa, Human immunodeficiency virus type 1 HXB2
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 117 aa, Artificial
  Alpha-body scAB013
MSIEEIQKQIAAIQKQIAAIQKQIYRMTGGSGGGSGGGSGGGSGMSIEEIQKQIAAIQKQIAAIQKQIYRMTGGSGGGSGGGSGGGSGMSIEEIQKQIAAIQKQIAAIQKQIYRMTP

SEQ ID NO: 3 - protein, 97 aa, Artificial
  scAB013_N1_L5/5
ALISGIQQQIANLQKAIAAQQHLIYLMTGGSSGGMSIEEIQKQIAAIQKQIAAIQKQIYRMTGGSSGGMSLSAIVKQQNAILKQIQAIQKQLQRMVA

SEQ ID NO: 4 - protein, 97 aa, Artificial
  scAB013_N2_L5/5
QQIANLQKAIAAQQHLIALIQWGIYRMTGGSSGGMSIEEIQKQIAAIQKQIAAIQKQIYRMTGGSSGGMSQNAILKQIQAIQKQLQAIVKQIKAMQA

SEQ ID NO: 5 - protein, 97 aa, Artificial
  scAB013_N3_L5/5
AAIAAQQHLIALIQWGIAQLQARIYAMTGGSSGGMSIEEIQKQIAAIQKQIAAIQKQIYRMTGGSSGGMSIQAIQKQLQAIVKQIKAIQKQILAMEA

SEQ ID NO: 6 - protein, 99 aa, Artificial
  scAB013_C1_L5/5
MSIEEIQKQIAAIQKQIAAIQKQIYRMTGGSSGGGGSSGGTWQMWEIQIQIYTIQIQILIIQAQIQQWKQMSIEEIQKQIAAIQKQIAAIQKQIYRMTP

SEQ ID NO: 7 - protein, 99 aa, Artificial
  scAB013_C2_L5/5
MSIEEIQKQIAAIQKQIAAIQKQIYRMTGGSSGGGGSSGGSMQMYTIQIQILIIQAQIQQIQNQIELMTLMSIEEIQKQIAAIQKQIAAIQKQIYRMTP

SEQ ID NO: 8 - protein, 6 aa, Artificial
  Linker sequence
GGSSGG

SEQ ID NO: 9 - protein, 40 aa, Human immunodeficiency virus type 1 HXB2
QLLSGIVQQQNNLLRAIEAQQHLLQLTVWGIKQLQARILA

SEQ ID NO: 10 - protein, 28 aa, Artificial
  Amino acid sequence of alpha-body helix
MSIEEIQKQIAAIQKQIAAIQKQIYRMT

SEQ ID NO: 11 - protein, 38 aa, Human immunodeficiency virus type 1 HXB2
HTTWMEWDREINNYTSLIHSLIEESQNQQEKNEQELLE

SEQ ID NO: 12 - protein, 28 aa, Artificial
  Alpha-body sequence
MLIEGIQKQIANIQKAIAAIQKLIYLMT

SEQ ID NO: 13 - protein, 28 aa, Artificial
  Alpha-body sequence
MQIENIQKAIAAIQKLIALIQKGIYQMT

SEQ ID NO: 14 - protein, 28 aa, Artificial
  Alpha-body sequence
MAIEAIQKLIALIQKGIAQIQKRIYAMT

SEQ ID NO: 15 - protein, 28 aa, Artificial
  Alpha-body sequence
MSISEIVKQINAILKQIEAIQKQIQRMV

SEQ ID NO: 16 - protein, 28 aa, Artificial
  Alpha-body sequence
MSINEILKQIEAIQKQIQAIVKQIKRMQ

SEQ ID NO: 17 - protein, 28 aa, Artificial
  Alpha-body sequence
MSIEEIQKQIQAIVKQIKAIQKQILRMT

SEQ ID NO: 18 - protein, 43 aa, Human immunodeficiency virus type 1 HBX2
QLLSGIVQQQNNLLRAIEAQQHLLQLTVWGIKQLQARILAVER

SEQ ID NO: 19 - protein, 28 aa, Artificial
  Amino acid sequence of structurally optimized alpha-body A-helix
ALISGIQQQIANLQKAIAAQQHLIYLMT

SEQ ID NO: 20 - protein, 28 aa, Artificial
  Amino acid sequence of structurally optimized alpha-body A-helix
QQIANLQKAIAAQQHLIALIQWGIYRMT

SEQ ID NO: 21 - protein, 28 aa, Artificial
  Amino acid sequence of structurally optimized alpha-body A-helix
AAIAAQQHLIALIQWGIAQLQARIYAMT

SEQ ID NO: 22 - protein, 29 aa, Artificial
  Amino acid sequence of structurally optimized alpha-body C-helix
MSLSAIVKQQNAILKQIQAIQKQLQRMVA

SEQ ID NO: 23 - protein, 29 aa, Artificial
  Amino acid sequence of structurally optimized alpha-body C-helix
MSQNAILKQIQAIQKQLQAIVKQIKAMQA

SEQ ID NO: 24 - protein, 29 aa, Artificial
  Amino acid sequence of structurally optimized alpha-body C-helix
MSIQAIQKQLQAIVKQIKAIQKQILAMEA

SEQ ID NO: 25 - protein, 28 aa, Artificial
  Amino acid sequence of alpha-body B-helix with grafted HR2 residues
WSIWDIQIQIYTIQIQILIIQSQIQQMT

SEQ ID NO: 26 - protein, 28 aa, Artificial
  Amino acid sequence of alpha-body B-helix with grafted HR2 residues
ISIYTIQIQILIIQSQIQQIQNQIELMT

SEQ ID NO: 27 - protein, 30 aa, Artificial
  Amino acid sequence of structurally optimized alpha-body B-helix
TWQMWEIQIQIYTIQIQILIIQAQIQQWKQ

SEQ ID NO: 28 - protein, 30 aa, Artificial
  Amino acid sequence of structurally optimized alpha-body B-helix
SMQMYTIQIQILIIQAQIQQIQNQIELMTL

SEQ ID NO: 29 - protein, 29 aa, Artificial
  Amino acid sequence of the non-mutated alpha-body C-helix
MSIEEIQKQIAAIQKQIAAIQKQIYRMTP

(19) United States
(12) Patent Application Publication
(10) Pub. No.: US 2012/0177676 A1
(43) Pub. Date: Jul. 12, 2012

(54) ALPHABODIES FOR HIV ENTRY INHIBITION

(75) Inventors: Johan Desmet, Kortrijk (BE); Ignace Joseph Isabella Lasters, Antwerpen (BE); Sophie Vanwetswinkel, La Roche (BE)

(73) Assignee: Complix NV, GentZwijnaarde (BE)

(21) Appl. No.: 13/382,647

(22) PCT Filed: Jul. 7, 2010
(86) PCT No.: PCT/EP10/59714

§ 371 Date: Mar. 26, 2012

(30) Foreign Application Priority Data
Jul. 8, 2009 (GB) 0911890.2

(51) Int. Cl. A61K 39/12; C12Q 1/70; A61K 38/16; C12N 5/10; C12N 15/13; A61P 37/00; C12N 1/19; C12N 1/21; C07K 14/00; A61P 31/18

(52) U.S. Cl. 424/186.1; 530/350; 435/5; 514/3.8; 536/23.5; 530/300; 435/254.23; 435/252.33; 435/325; 435/349; 435/419; 435/348

(57) ABSTRACT

The present invention relates to HIV-1 gp41-binding single-chain 3-stranded alpha-helical coiled coil molecules, denoted “Alphabodies”, nucleic acids encoding said Alphabodies, host cells comprising said nucleic acids, as well as pharmaceutical compositions comprising said Alphabodies, and methods for the treatment, prevention and diagnosis of HIV infection using said Alphabodies.

### FIELD OF THE INVENTION

The present invention relates to the field of human immunodeficiency virus (HIV) and treatment of HIV infections. More specifically, the present invention relates to a new class of HIV entry inhibitors. The present invention further relates to the usage of single-chain 3-stranded alpha-helical coiled coil molecules, denoted “Alphabodies”, for usage as HIV entry inhibitors targeting gp41 sub-regions.

### BACKGROUND OF THE INVENTION

HIV-1 Env complexes, also known as “envelope glycoprotein complexes” or “gp120/gp41 complexes” or “HIV spikes”, are a primary target for treatment of HIV infection. They are displayed at the surface of HIV virions and cells that are engineered so as to express envelope spikes. HIV entry into a target cell and cell-cell fusion are primarily mediated by the action of Env glycoprotein complexes displayed at the surface. The ability to block viral entry or cellular fusion is generally thought to be of high value for the treatment of HIV infection.

Inhibition of viral entry can be accomplished by antiviral compounds that specifically target sub-regions of gp120, gp41 or fusion-mediating receptors CD4, CCR5 or CXCR4. Compounds that specifically target gp41, such as enfuvirtide (Fuzeon, T-20), are intended to exert their antiviral effect through blockage of a key-step in the fusion mechanism. This key-step is commonly known as the formation of a 6-helix bundle. 6-Helix bundles are formed as a result of gp41-HR1 fragments forming a trimeric parallel coiled coil structure and -HR2 fragments binding tightly onto this coiled coil. Since HR1 can attach to a target cell membrane via the N-terminally located fusion peptide, whereas HR2 is attached to the viral membrane via the C-terminally located transmembrane fragment, condensation of the ectodomain of the gp41 chain into a compact 6-helix bundle is believed to bring the two membranes in close proximity, promote membrane fusion and, hence, entry of the virion (or rather its content) into the target cell.

### SUMMARY OF THE INVENTION

The present invention relates to a new class of HIV entry inhibitors that specifically address (target) sub-regions in gp41, for the purpose of blocking 6-helix bundle formation. This new class of inhibitors corresponds to a single-chain coiled coil protein scaffold, denoted an “Alphabody”, that is engineered to bind either to gp41-HR1 (or a fragment nearby in the sequence) or gp41-HR2 (or a nearby fragment). The present invention also relates to a bifunctional Alphabody that can simultaneously bind to gp41 regions HR1 and HR2, or regions nearby. Further, the present invention relates to the usage of such Alphabodies for inhibition of HIV Env-mediated cell-cell fusion, HIV viral entry inhibition, viral replication inhibition, treatment of HIV infection in mammalian and human cells, a method of treating HIV-infected individuals, a method to screen for novel anti-HIV compounds using an Alphabody, a method known as a competition assay, and a method for vaccinating an individual against HIV.

The present invention relates to the usage of a single-chain 3-stranded coiled coil scaffold, denoted “Alphabody” that is engineered for the purpose of binding to HIV gp41 sub-regions such as HR1, HR2 and adjacent sub-regions in a gp41 amino two distinct sub-regions (bifunctional, bispecific binding). Practical examples are provided to generate such Alphabodies. The present invention also relates to methods for inhibiting HIV infection in an individual. Further, Alphabodies of the present invention are proposed as useful screening tools to identify novel HIV-inhibiting molecules, either through a direct binding assay or through a competition assay. Finally, methods are proposed to administer Alphabodies of the present invention as a vaccine or a medicament.

### BRIEF DESCRIPTION OF THE DRAWINGS

FIG. 1. Formation of a 6-helix bundle composed of HR1- and HR2-derived peptides. White cylinders, representation of gp41-HR1-derived peptidic fragments forming a trimeric parallel coiled coil (N-trimer); gray cylinders, representation of gp41-HR2-derived peptidic fragments binding to the grooves in between pairs of HR1-helices. Labels “N” and “C” denote the N- and C-terminal ends of the helices, respectively. Thus, HR2 helices are bound in antiparallel orientation relative the HR1 helices. The result of the binding is a 6-helix bundle, as depicted to the right of the white arrow.

FIG. 2. Formation of a 6-helix bundle composed of HR1- and HR2-fragments from gp41, interconnected by a loop region. Shading and labeling is as in FIG. 1. Curved arrows represent the loop region (disulfide loop, hairpin loop) connecting HR1 fragments (white cylinders) to HR2 fragments (gray cylinders) in HIV Env spikes prior to formation of the 6-helix bundle (to the left of the white arrow) and after formation of the 6-helix bundle (at the right). The mutual orientation of HR1 and HR2 helices in native or receptor-activated spikes is not necessarily the same as depicted in the left panel; the latter intends to illustrate that HR1 and HR2 fragments are covalently interconnected yet separated in space.

FIG. 3. Parallel and antiparallel Alphabodies. Panel A, parallel Alphabody; panel B, antiparallel Alphabody. Parallel helices are represented by white cylinders, whereas the helix that is antiparallel to the two others in panel B is depicted as a gray cylinder. Curved arrows connecting different helices represent linker fragments. The tiny non-connecting arrows represent N- and C-terminal extensions to the Alphabody. Helices are labeled A, B, C according to their appearance in the Alphabody, which is composed of a single-chain amino acid sequence.

FIG. 4. Binding of an Alphabody to gp41 HR2. Shading and labeling is as in FIGS. 2 and 3. The Alphabody is represented by bold cylinders. This figure illustrates that an Alphabody bound to an HR2 fragment of gp41 prevents the latter from binding to the gp41 N-trimer and thereby precludes formation of a 6-helix bundle. The possibility that two or three HR2 fragments in an HIV spike are bound by an equal number of Alphabodies is not shown in the figure, but also not excluded by it.

FIG. 5. Binding of an Alphabody to gp41 HR1. Shading and labeling is as in FIG. 4. This figure illustrates that an Alphabody bound to a gp41 N-trimer prevents the latter from binding to a gp41 HR2 fragment and thereby precludes formation of a 6-helix bundle. The possibility that two or three Alphabodies bind to an equal number of N-trimer grooves of an HIV spike is not shown in the figure, but also not excluded by it.

FIG. 6. Simultaneous binding of an Alphabody to gp41 HR1 and HR2. Shading and labeling is as in FIG. 4. This figure illustrates that an Alphabody bound simultaneously to a gp41 N-trimer and to a gp41 HR2 fragment prevents the latter from binding to the N-trimer and thereby precludes formation of a 6-helix bundle. This figure thus illustrates a bifunctional Alphabody. The possibility that two or three Alphabodies bind in a similar way to an HIV spike is not shown in the figure, but also not excluded by it.

FIG. 7. Structural aspects related to residue grafting. Panel A shows a representation of an N-trimer and an HR2 helix. In this example, the N-trimer is taken to be the N40 sequence as published in Root et al. Science 2001, 291: 884-888. The HR2 sequence is taken to be a C38 sequence (ibid). Helices are looked upon with the N-trimer Z-axis pointing backward (N-terminus in front) and with the HR2 helical axis pointing forward (N-terminus at the back). Thus, the encircled helices are antiparallel to each other. The small arrow between the circles suggests a superposition of an HR2 (C-)helix on top of an N-helix, without considering the mismatch in direction. In practice this would correspond to mapping of HR2 positions c, f, b, e, a, d, g onto N-helix positions d, a, e, b, f, c, g, respectively. Replacing the N-helix by the fitted C-helix would give a construct similar to the one depicted in panel B. However, an immediate and serious problem of this way of recombining N- and C-peptide fragments is the inevitable disturbance of the core packing. Key to the present invention is that such problem does not exist when using antiparallel Alphabodies. It is sufficient to graft the HR2 positions labeled d, a and a in panel A onto positions labeled b, f and c in panel B, respectively, to obtain an Alphabody construct with a well-packed isoleucine-core and which displays both an HR2-binding groove and a helix carrying the groove-binding HR2 residues. Panels C and D illustrate how such construct could capture the respective regions in a viral gp41 molecule; the views in panels C and D are along and perpendicular to the helical axes, respectively (all helices are idealized and the supercoiling is ignored). The double arrows symbolize nonbonded interactions (binding). The long arrow connecting an N-helix with an HR2 helix from the virus represents the hairpin loop. Panel D clearly illustrates that helix B in the Alphabody (with the grafted HR2 residues) and a pair of helices from the viral N-trimer (forming the binding site) are antiparallel. Likewise, the groove-forming helices A and C of the Alphabody and the viral HR2 helix are also antiparallel. This figure thus explains the rationale that lies at the basis of the construction of a bispecific Alphabody with the potential to simultaneously target the N-trimer and an HR2 fragment in HIV-1 Env spikes.

FIG. 8. Sequence alignments of an Alphabody with HR1 and HR2 sequences. A, alignment of HR1 sequence denoted “N-40” (residues 543 to 582 of gp160 HXB2) with the sequence of selected Alphabody denoted “scAB013” (only 1 helix thereof) in three different frames. Heptad a/d-positions are shaded in gray. B, alignment of HR2 sequence denoted “C38” (residues 625 to 662 of gp160 HXB2) with the sequence of selected Alphabody scAB013 (1 helix) in two different frames, such that C38 a-, d- and e-positions map onto the Alphabody f-, b- and c-positions, respectively. In this way, the HR2 (C38) contact residues (shaded in gray), when grafted onto the Alphabody, would face away from the center of the Alphabody.

FIG. 9. Initial selection of groove amino acids. A, selection of residues to be grafted at g- and c-positions of an Alphabody A-helix (shaded in gray) in the three registers that are possible for Alphabody/HR1 alignment. B, amino acid sequence of the non-mutated Alphabody B-helix, supplemented with appropriate flanking linkers L1 and L2. C, selection of residues to be grafted at e- and b-positions of an Alphabody C-helix (shaded in gray) in the three possible heptad registers.

FIG. 10. Structurally optimized groove sequences. A, amino acid sequence of the structurally optimized A-helix in the three registers that are possible for Alphabody/HR1 alignment. B, amino acid sequence of the non-mutated Alphabody B-helix, supplemented with appropriate flanking linkers L1 and L2. C, amino acid sequence of the structurally optimized C-helix in the three possible alignment registers. Residues appearing in the reference Alphabody scAB013 are not underlined. Singly underlined residues are grafted from HR1 amino acids. Doubly underlined residues are mutations selected on basis of structural considerations.

FIG. 11. Initial grafting of HR2 residues. B, amino acid sequence of Alphabody B-helix with grafted HR2 residues (shaded in gray) in two possible registers for alignment to HR2 peptide C38, The label “B.” only serves to indicate that grafting is to be performed in the Alphabody B-helix.

FIG. 12. Structurally optimized B-helix sequences. A, amino acid sequence of the non-mutated Alphabody A-helix, supplemented with an appropriate flanking linker L1. B, structurally optimized B-helix in the two registers that are possible for Alphabody/HR2 alignment. C, amino acid sequence of the non-mutated Alphabody C-helix, preceded with an appropriate flanking linker L2. Residues appearing in the reference Alphabody scAB013 are not underlined. Singly underlined residues are grafted from HR2 amino acids. Doubly underlined residues are mutations selected on basis of structural considerations.

FIG. 13. Final Alphabody constructs with N-trimer-like binding grooves. A, 3 amino acid sequences to be incorporated as the A-helix in an Alphabody; L1, linker 1 sequence; B, amino acid sequence to be incorporated as the B-helix in an Alphabody, irrespective of A- and C-helix sequences; L2, linker 2 sequence; C, 3 amino acid sequences to be incorporated as the C-helix in an Alphabody. Residues are underlined according to the same conventions as in FIG. 10. The sequences are to be concatenated in an Alphabody in the order Ai-L1-B-L2-Ci, where i refers to any of the indices preceding the sequences under A and C. Thus, this figures represents three different constructs that are designed to target different sub-regions of HR2 in HIV-1 gp41.

FIG. 14. Final Alphabody constructs with HR2-like interface residues. A, amino acid sequence to be incorporated as the A-helix in an Alphabody, irrespective of the B-helix sequence; L1, linker 1 sequence; B, 2 amino acid sequences to be incorporated as the B-helix in an Alphabody; L2, linker 2 sequence; C, amino acid sequence to be incorporated as the C-helix in an Alphabody, irrespective of the B-helix sequence; Residues are underlined according to the same conventions as in FIG. 12. The sequences are to be concatenated in an Alphabody in the order A-L1-Bi-L2-C, where i refers to any of the indices preceding the sequences under B. Thus, this figures represents two different constructs that are designed to target different sub-regions of the N-trimer in HIV-1 gp41.

FIG. 15. CD thermoscans of scAB013_N3 in different GuHCl concentrations. The Alphabody was dissolved in 20 mM PBS, 150 mM NaCl, pH 7.2 and in concentrations of GuHCl as indicated: diamonds, 2 M; squares, 4 M; circles, 6 M of denaturant. The Alphabody concentration was approximately 12 microM. The mean residual ellipticity ([Theta]) was recorded at a wavelength of 222 nM. Closed symbols correspond to upward (heating) scans and open symbols to downward (cooling) scans.

FIG. 16. Isothermal titration calorimetry (ITC) of scAB013_N3 titrated with C36 derivative bL4_C36. The cell was filled with 2.46 microM of scAB013_N3 in 20 mM PBS, 150 mM NaCl, pH 7.2 and the injection syringe was filled with 50 microM bL4_C36 in the same buffer. FIG. 16A shows the raw thermogram after correction for baseline drift. The peaks were integrated, corrected for friction/dilution effects by subtracting the mean of the last 4 peaks, and then integrated again to obtain the cumulative enthalpy change (ΔH) of FIG. 16B, wherein the latter is plotted as a function of the molar ratio bL4_C36 over scAB013_N3. Curve fitting using an equilibrium model with 1:1 binding stoechiometry yielded the thermodynamic parameters ΔH=−30 kJ/mol and Kd=150 nM.

### DETAILED DESCRIPTION OF THE INVENTION

The human immunodeficiency virus type 1 (HIV-1) envelope glycoprotein complex (HIV-1 Env, spike) consists of a trimer of glycoprotein 120 (gp120) and glycoprotein 41 (gp41) heterodimers. Such spikes are natively displayed in a prefusion state. Upon attachment to a target cell, mediated by cellular receptors CD4 and chemokine coreceptors (CXCR4 or CCR5), certain conformational changes occur, leading eventually to the postfusion state and concomitant membrane fusion. Intermediate to the prefusion and the postfusion states, and subsequent to partial conformational changes, the spikes adopt a state known as the pre-hairpin state or fusion intermediate. Although little is known about the structural nature of the pre-hairpin state, or states, the latter are considered important subjects for targeting because certain sub-regions that are hidden in the pre- and postfusion states then become accessible to inhibitor molecules.

Sub-regions within HIV-1 Env spikes that have attracted specific attention are the fragments known as heptad repeat 1 (HR1) and heptad repeat 2 (HR2). Upon transition from the pre- to the postfusion state, HR1 fragments form a trimeric (3-stranded) parallel coiled coil structure, also known as “N-trimer”. This coiled coil structure is composed of three tightly interacting parallel alpha-helices. Along these alpha-helices, and in between each helix pair, there is a shallow groove. Thus, there are three grooves per N-trimer. Since the HR1 amino acid sequences in each spike are identical, the N-trimer is expected to be in principle structurally symmetrical, although during the dynamic process of conformational changes from the pre- to the postfusion state this structural symmetry will presumably not be preserved at each moment.

As known from experimentally determined three-dimensional (3-D) structures representing the postfusion state, each of the three grooves in an N-trimer has at least the capacity to bind an HR2 fragment. Thus, there are three HR2-binding grooves (sites, interfaces) per N-trimer. The same 3-D structures also show that HR2 fragments bind in an alpha-helical conformation and these alpha-helices are bound in an antiparallel orientation relative to the HR1 helices of the N-trimer. The HR1 helices form the inner trimeric parallel coiled coil and the HR2 helices form the outer helices.

A complex between HR1- and HR2-derived peptides is generally denoted a “six-helix bundle” (see FIG. 1). The latter can be formed by simple mixing of HR1- and HR2-derived peptides, also known as N- and C-peptides, respectively. However, within the context of HIV spikes, the HR1- and HR2-fragments do not exist as free peptides. There, they form sub-regions within the contiguous amino acid chain known as glycoprotein 41 (gp41). The latter has been subdivided into functional regions as follows (see, for example, Noah et al. Biochemistry 2008, 47:6782-6792): (i) fusion peptide (FP, residues 512 to 527), fusion peptide-proximal region (FP-PR, residues 528 to 540), N-peptide region or heptad repeat 1 (HR1, residues 541 to 581), disulfide loop or hairpin-forming loop or loop region (LR, residues 582 to 627), C-peptide region or heptad repeat 2 (HR2, residues 628 to 665), membrane proximal external region (MPER, residues 666 to 683), transmembrane region (TM, residues 684 to 705) and intracellular or cytoplasmic region (CP, residues 706 to 856). The residue numbering is herein based on gp160 of HIV-1 HXB2. In different publications, the boundaries to said functional regions can differ up to about 5 residues. The extracellular domain (ectodomain) of gp41 corresponds to residues 512 to 683. Thus, HR1 and HR2 are respectively located within the first (N-terminal) and second (C-terminal) half of the gp41 ectodomain, and they are separated by the loop region.

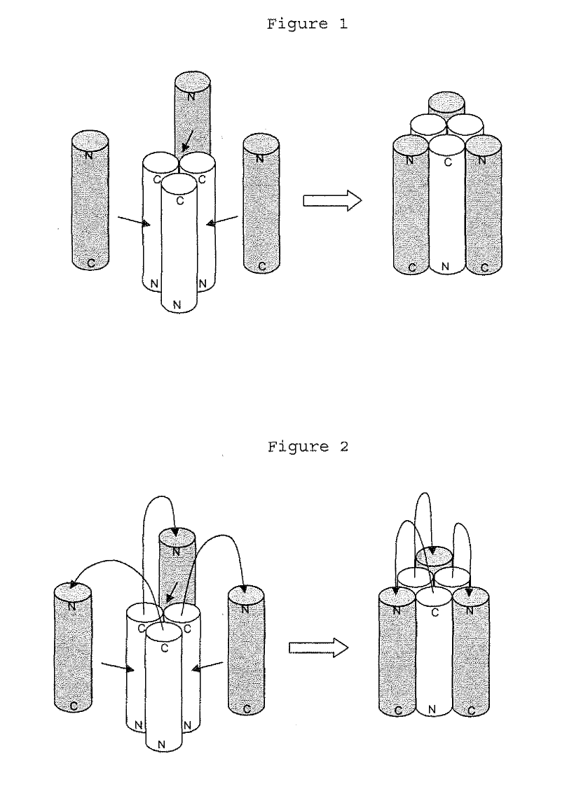

Within the context of the gp41 ectodomain, the fragments HR1 and HR2 can form a 6-helix bundle that is highly similar in structure to peptidic 6-helix bundles. Then, a six-helix bundle consists of a trimer of HR1/HR2 heterodimers, also known as a trimer of hairpins, and wherein each HR1 is linked to HR2 by a loop region (see FIG. 2). Thus, the loop region fulfills a dual role: that of a linker and a separator.

It is not known in the art how exactly HR1 and HR2 are kept apart in native, prefusion spikes. Yet, it is known that a spike exists as a trimer of gp120/gp41 heterodimers (not to be confused with the HR1/HR2 heterodimers). There is only scarce data on which residues from gp120 and gp41 form the interface between the two types of subunits in the prefusion state, and there is no direct structural evidence at all. Yet, there is growing evidence that different sub-regions from gp41 are involved in the interaction with gp120 and that these sub-regions bind more or less independently from each other (see, e.g., Kim et al. J Mol Biol 2008, 376:786-797). It is therefore possible that gp120 acts as a spatial separator for gp41 fragments in the prefusion state. Further, it is known that gp41 inserts into a target cell via its fusion peptide that is connected to HR1 through the FP-PR. The latter leads to the prefusion intermediate state wherein HR1 is necessarily located distal (separated in space) from HR2 which is itself attached to the viral membrane through the MPER and TM regions. All conformational changes are triggered by CD4 and/or coreceptor binding to gp120 (and not gp41). It is therefore believed that there exists a “window of opportunity” during the fusion mechanism wherein the otherwise occluded HR1 and HR2 fragments become accessible for targeting and subsequent inhibition of the fusion process.

The functional role of the HR1 and HR2 fragments in gp41 is widely believed to drive fusion of viral and target cell membranes through formation of the 6-helix bundle. Thus, inhibition of 6-helix bundle formation is considered a valid strategy to prevent membrane fusion and viral infection.

Of special interest with respect to the present invention are certain peptides that are directly derived from gp41 HR1 and HR2 sequences. For example, HR2-derived peptides such as C34 (res. 628-661) are thought to directly target the N-trimer where they form a steric block for HR2, thus preventing 6-helix bundle formation and membrane fusion. Reversely, one of the proposed mechanisms of action for N-peptides, e.g. N36 (res. 546-581), is that they form trimeric complexes in solution that are able to trap HR2 when it becomes accessible. Although the latter possibility remains a matter of debate, it has nevertheless stimulated the design of the 5-helix bundle (see, e.g., Root et al. Science 2001, 291:884-888 and patent application US2006/0014139 A1). In addition, other researchers have designed stabilized N-trimer constructs (e.g., NCCG-gp41, N35CCG-N13, IQ-N23, see references in Gustchina et al. J Virol 2008, 82:10032-10041). All of them were shown to be strongly antiviral, with IC50s typically in the low-nanomolar range. Consequently, both the HR1- and HR2-subfragments were demonstrated to be validated target regions within HIV-1 gp41.

Various HIV entry-inhibiting molecules (entry or fusion inhibitors) have been developed in the past, with variable success. They can grossly be subdivided into antibodies, non-immunoglobulin proteins, peptides, and small molecules. Nonlimiting examples of monoclonal antibodies are D5, 2F5 and 4E10. Nonlimiting examples of non-immunoglobulin proteins are soluble CD4 and 5-helix. Nonlimiting examples of peptides are T20, T-1249 and N36. Nonlimiting examples of small molecules are BMS-806, Maraviroc and AMD070. The only entry inhibitor approved so far for clinical use is enfuvirtide (Fuzeon, T20). Yet, the latter drug suffers from problems relating to safety, tolerability, injection site reactions, patient compliance, and the development of resistance. Accordingly, there is a continuing need for new anti-HIV drugs including HIV entry inhibitors. There is also a continuing need for new drugs and drug leads with improved characteristics compared to existing antiviral drugs and leads. More specifically, there is a need for peptidic or proteinaceous drugs that inhibit HIV entry and cell-cell fusion with improved activity (antiviral potency, antiviral efficacy, at lower dosages, at lower EC50 values). There is also a need for proteinaceous drugs that are smaller in molecular size than, for example, antibodies or 5-helix; such drugs can likely be produced at lower cost, require lower amounts of material (for a constant molar EC50), can potentially be administered topically (i.e., in creams, patches) and are likely more suitable to target partially occluded binding regions in spikes (i.e., they are likely less susceptible to steric hindrance effects and have a higher diffusion coefficient). Further, there is also a need for stable drugs with a long shelf life, high solubility and protease resistance. Most of all, there is a high need for new drugs with reduced propensity to elicit escape mutations (resistance mutations).

All embodiments of the present invention relate to single-chain coiled coil molecules which are herein collectively denoted “Alphabodies”. Similar single-chain coiled coils have been described in Desmet et al, EP 08172017.9 and Desmet et al., U.S. 61/120,642. Briefly, an Alphabody shall herein mean a single-chain coiled coil having a single contiguous amino acid chain with the formula HRS1-L1-HRS2-L2-HRS3, optionally supplemented with N- and C-terminal extensions resulting in the formula N-HRS1-L1-HRS2-L2-HRS3-C, wherein (a) each of HRS1, HRS2 and HRS3 is independently a heptad repeat sequence (HRS), consisting of 2 to 7 consecutive heptad repeat (HR) units, which sequence can be designated as a-b-c-d-e-f-g, at least 50% of all heptad a- and d-positions are occupied by isoleucine residues, and HRS1, HRS2 and HRS3 together constitute a 3-stranded alpha-helical coiled-coil structure; (b) each of L1 and L2 are independently a linker fragment, covalently connecting HRS1 to HRS2 and HRS2 to HRS3, respectively, starting and ending with a praline or glycine, and consisting of 3 to 30 amino acid residues of which at least 50% are selected from the group proline, glycine, serine; and (c) N and C are independently an optional extension, covalently connected to the N- and C-terminal end of HRS1 and HRS3, respectively, this connection being marked by a helix-breaking proline or glycine.

As stated above, at least 50% of all heptad a- and d-positions are occupied by isoleucine residues. The remaining a- and d-positions can be any of the 20 naturally occurring amino acids, or non-naturally occurring amino acids.

Furthermore, the amino acids in each of L1 and/or L2 that are not proline, glycine, or serine can be any of the 20 naturally occurring amino acids, or non-naturally occurring amino acids.

Amino acids at positions b, c, e, f and g can also be any of the 20 naturally occurring amino acids, or non-naturally occurring amino acids.

The term “naturally occurring amino acid” refers to the following amino acids: alanine, aspartic acid, asparagine, cysteine, glutamine, glutamic acid, phenylalanine, glycine, histidine, isoleucine, lysine, leucine, methionine, proline, arginine, serine, threonine, valine, tryptophan, and tyrosine.

The term “non-naturally occurring amino acid” as used herein, refers to amino acids having a side chain that does not occur in the naturally occurring L-amino acids. Examples of non-natural amino acids and derivatives include, but are not limited to, agmatine, (S)-2-amino-4-((2-amino)pyrimidinyl)butanoic acid, 4-amino butyric acid, 4-amino-3-hydroxy-5-phenylpentanoic acid, 4-amino-3-hydroxy-6-methylheptanoic acid, 6-aminohexanoic acid, alpha-aminoisobutyric acid, benzophenone, t-butylglycine, citrulline, cyclohexylalanine, desamino tyrosine, L-(4-guanidino)phenylalanine, homoarginine, homocysteine, homoserine, homolysine, n-formyl tryptophan, norleucine, norvaline, phenylglycine, (S)-4-piperidyl-N-amidino)glycine, ornithine, parabenzoyl-L-phenylalanine, sarcosine, statine, 2-thienyl alanine, and/or D-isomers of the naturally or non-naturally occurring amino acids.

Alphabodies are relatively small in size (about 10 to 20 kDa). Accordingly, this property is in agreement with the need for therapeutic protein molecules of a size that is smaller than an antibody or 5-helix. Alphabodies are also highly thermostable and are relatively insensitive to changes in pH and to proteolytic degradation. These properties form a solid basis for the development of engineered Alphabodies with preservation of desirable physico-chemical properties and with acquired therapeutic functions. Therefore, Alphabodies are in agreement with the need for therapeutic molecules that have a long shelf life. Alphabodies are also highly soluble, which is in agreement with the need for therapeutic molecules that can be easily tested in vitro. Most importantly, the fact that Alphabodies are highly engineerable (substitutable, mutatable) is in agreement with the need for generating novel therapeutic molecules with high-affinity and specificity for selected target molecules.

In general, Alphabodies are well suited as scaffold molecules for target recognition, for they are relatively insensitive to multiple simultaneous amino acid substitutions. For example, the structural integrity of an Alphabody is in general not substantially affected when all amino acid residues of a single groove are simultaneously mutated. Similarly, the structural integrity does not substantially change when all surface-exposed amino acid residues of a single alpha-helix are simultaneously mutated.

Accordingly, applicants have contemplated the possibility of introducing multiple substitutions into a reference Alphabody with the aim of providing an Alphabody that binds to the extracellular domain of HIV-1 gp41, said extracellular domain being defined as amino acid residues 1 to 683 of SEQ ID NO: 1.

Hence, the present invention relates to an Alphabody which binds to the HR1 fragment of HIV-1 gp41, said HR1 fragment being defined as amino acid residues 546 to 581 of SEQ ID NO: 1.

The present invention also relates to an Alphabody which binds to the HR2 fragment of HIV-1 gp41, said HR2 fragment being defined as amino acid residues 628 to 661 of SEQ ID NO: 1.

The present invention also relates to an Alphabody which binds simultaneously to the HR1 and HR2 fragments of HIV-1 gp41, said HR1 and HR2 fragments being defined as amino acid residues 546 to 581 and 628 to 661 of SEQ ID NO: 1, respectively.

Further, the present invention also relates to Alphabodies displaying a binding site for gp41 sub-regions near HR1, i.e., the fusion peptide, the fusion peptide-proximal region, and the first half of the loop region. Analogously, the provision of Alphabodies displaying a binding site for gp41 sub-regions near HR2, i.e., the second half of the loop region and the membrane-proximal external region are included within the invention, as well as Alphabodies displaying both a binding site for gp41 sub-regions near HR1 and near HR2, thus bifunctional Alphabodies.

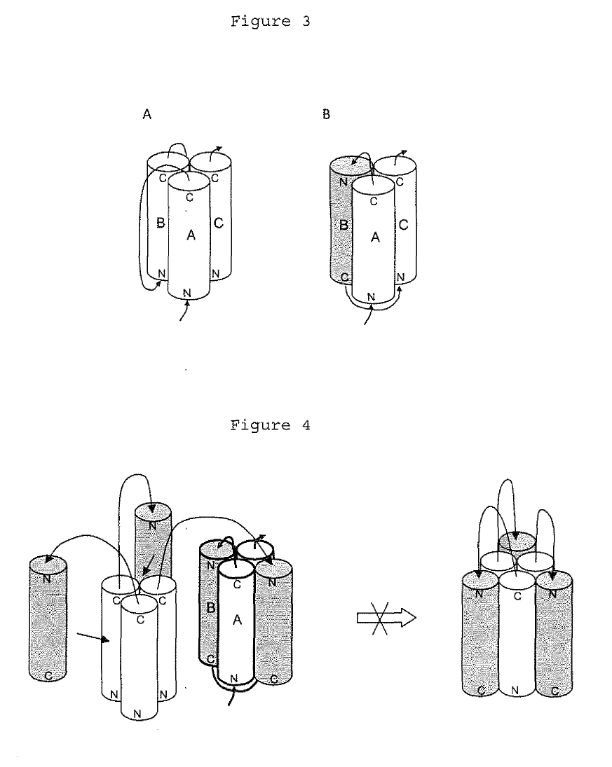

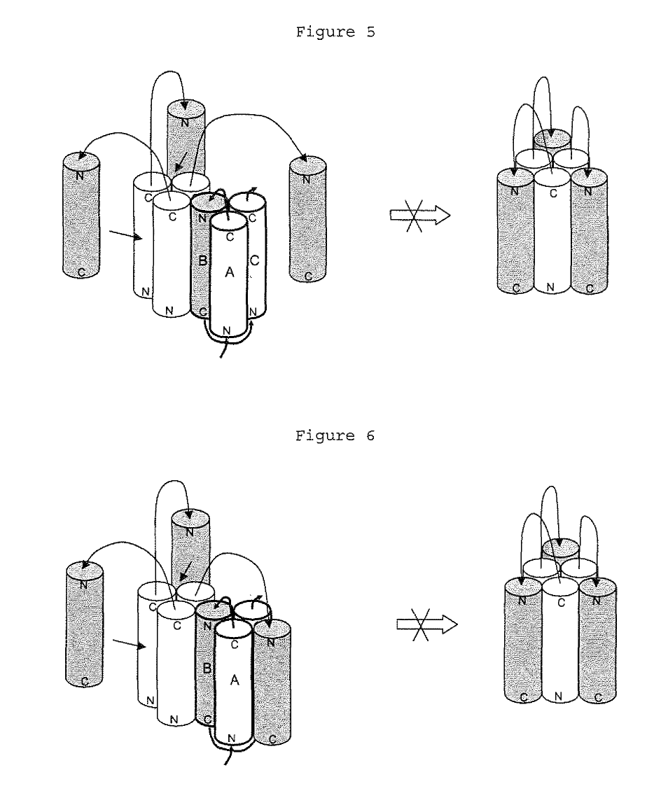

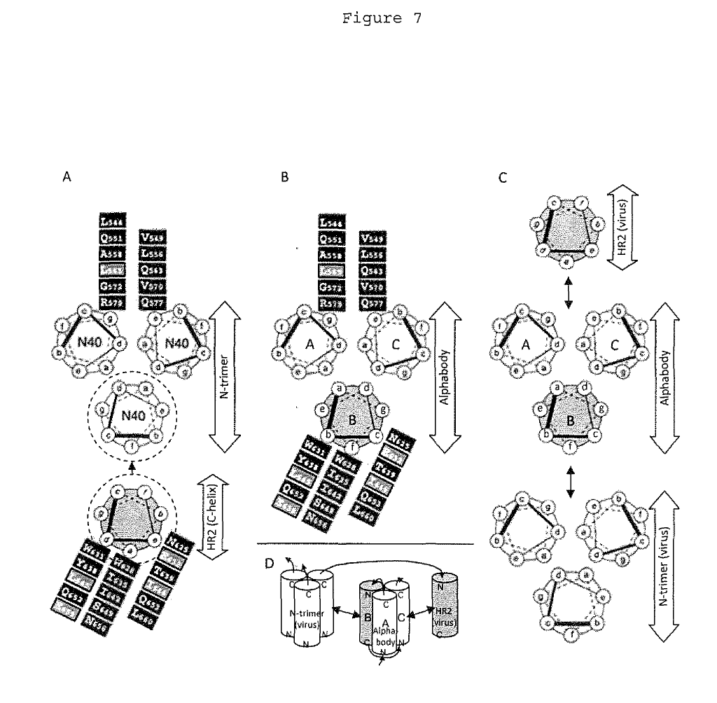

Alphabodies can exist as either parallel or antiparallel single-chain coiled coils (FIG. 3). Both parallel and antiparallel Alphabodies are suited for targeting sub-regions within gp41. The direct functional effect of binding to such sub-regions is anticipated to be the blocking (for example, prevention or inhibition) of 6-helix bundle formation. Such blocking by binding to gp41 sub-regions near HR2 is illustrated in FIG. 4. Such blocking by binding to gp41 sub-regions near HR1 is illustrated in FIG. 5. Such blocking by binding simultaneously to gp41 sub-regions near HR1 and HR2 is illustrated in FIG. 6.

In view of the fact that HR2 fragments in a 6-helix bundle are antiparallel relative the helices of the central N-trimer, applicants have contemplated that antiparallel Alphabodies may be better suited than parallel Alphabodies to simultaneously bind to gp41 sub-regions near HR1 and HR2, although parallel Alphabodies may provide bifunctional binding as well.

One advantage of bifunctional binding may be a higher binding affinity or a more potent antiviral effect. Another advantage of bifunctional binding may be a lower propensity for eliciting resistance mutations. Accordingly, certain embodiments of the present invention may be in agreement with the need for new antiviral drugs that are less susceptible to viral resistance.

In a preferred embodiment, Alphabodies are provided wherein the binding to HIV-1 gp41 is characterized by a dissociation constant (Kd) or half maximal effective concentration (EC50) in the submicromolar range, preferably a dissociation constant (Kd) or half maximal effective concentration (EC50) of less than 1.0 micromolar, or the subnanomolar range, preferably a dissociation constant (Kd) or half maximal effective concentration (EC50) of less than 1.0 nanomolar, or the subpicomolar range, preferably a dissociation constant (Kd) or half maximal effective concentration (EC50) of less than 1.0 picomolar. These techniques include, but are not limited to RIA (radioimmunoassays), ELISA (enzyme-linked immunosorbent assays), “sandwich” immunoassays, immunoradiometric assays, gel diffusion precipitation reactions, immunodiffusion assays, Western blots, precipitation reactions, agglutination assays (e.g. gel agglutination assays, hemagglutination assays, etc.), complement fixation assays, immunofluorescence assays, protein A assays, and immunoelectrophoresis assays, etc.

As used herein, the term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the Alphabodies may be detected by various methods, including the use of radiolabeled antibodies, enzyme linked antibodies, etc.

The binding specificity of Alphabodies of the current invention can be determined by an in vitro binding assay, such as radioimmunoassay (RIA) and enzyme-linked immunosorbent assay (ELISA). Such techniques and assays are known in the art. The binding affinity of the Alphabodies can, for example, be determined by Scatchard analysis, Friquet analysis, surface plasmon resonance or isothermal titration. It is advantageous to identify Alphabodies having a high degree of specificity and a high binding affinity for the target HIV env antigen.

In another preferred embodiment, Alphabodies are provided wherein the binding to HIV-1 gp41 inhibits HIV Env-mediated cell-cell fusion, characterized by a half maximal inhibitory concentration (IC50) in the submicromolar range, preferably a half maximal inhibitory concentration (IC50) of less than 1.0 micromolar, or the subnanomolar range, preferably a half maximal inhibitory concentration (IC50) of less than 1.0 nanomolar, or the subpicomolar range, preferably a half maximal inhibitory concentration (IC50) of less than 1.0 picomolar, said inhibition of HIV Env-mediated cell-cell fusion being measured by a cell-cell fusion assay.

In another preferred embodiment, Alphabodies are provided wherein the binding to HIV-1 gp41 inhibits HIV viral entry, characterized by a half maximal inhibitory concentration (IC50) in the submicromolar range, preferably a half maximal inhibitory concentration (IC50) of less than 1.0 micromolar, or the subnanomolar range, preferably a half maximal inhibitory concentration (IC50) of less than 1.0 nanomolar, or the subpicomolar range, preferably a half maximal inhibitory concentration (IC50) of less than 1.0 picomolar, said inhibition of HIV viral entry being measured by a single-cycle antiviral infection assay.

In another preferred embodiment, Alphabodies are provided wherein the binding to HIV-1 _{gp}41 inhibits HIV viral replication, characterized by a half maximal inhibitory concentration (IC50) in the submicromolar range, preferably a half maximal inhibitory concentration (IC50) of less than 1.0 micromolar, or the subnanomolar range, preferably a half maximal inhibitory concentration (IC50) of less than 1.0 nanomolar, or the subpicomolar range, preferably a half maximal inhibitory concentration (IC50) of less than 1.0 picomolar said inhibition of HIV viral replication being measured by a viral replication assay.

In another preferred embodiment, Alphabodies are provided wherein the binding to HIV-1 gp41 inhibits HIV infection of mammalian cells in vitro or in vivo.

In another preferred embodiment, Alphabodies are provided wherein the binding to HIV-1 gp41 inhibits HIV infection of human cells in vitro or in vivo.

In yet another preferred embodiment, a method of inhibiting HIV infection in an individual is provided, comprising administering to the individual an HIV-1 gp41-binding Alphabody according to the invention.

In yet another preferred embodiment, the HIV-1 gp41-binding single-chain coiled coil according to the invention is for use in the treatment of HIV infection.

As used herein, a person skilled in the relevant art may generally understand the term “treatment” to generally refer to an approach for obtaining beneficial or desired results. Beneficial or desired results can include, but are not limited to, prevention or prophylaxis, alleviation or amelioration of one or more symptoms or conditions, diminishment of the extent of a disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, a person skilled in the relevant art may generally understand the term “therapeutically effective amount” to be an amount sufficient to effect treatment when administered to a subject in need of treatment. In the case of the embodiments of the present invention, a therapeutically effective amount can include, but is not limited to, an amount that eliminates or reduces the effects of the disease in a subject.

The invention also relates to a pharmaceutical composition comprising an HIV-1 gp41-binding single-chain coiled coil as described above according to the invention and a pharmaceutically acceptable carrier.

It will be understood by a person skilled in the relevant art that modifications of the Alphabodies of the present invention are contemplated herein. The Alphabodies of the present invention may be modified by conjugating, tagging or labeling through methods known in the art, to any known diagnostic or therapeutic agent, including but not limited to cytotoxic agents (e.g. immunotoxin conjugates), prodrugs, drugs (e.g. pharmaceutically active substances) or other effector molecules which are effective in the treatment of disease as well as known reporter molecules. Such modified Alphabodies include, but are not limited to (a) labeled (e.g. radiolabeled, enzyme-labeled, fluorochrome or chemiluminescent compound) Alphabodies of the present invention, for diagnostic purposes using known imaging technologies and (b) immunotoxin conjugates of the Alphabodies of the present invention, where the Alphabodies of the present invention are conjugated to known cytotoxic, radioactive, radiolabeled, prodrug or drug moieties (e.g. radioimmunotherapy). It will be understood by a person skilled in the relevant art that the term “cytotoxic agent”, “cytotoxins” or “cytotoxic” as used herein generally refer to a substance that inhibits or prevents the function of cells and/or causes destruction of cells and includes, but is not limited to, radioactive isotopes, chemotherapeutic agents, and toxins such as small molecule toxins or proteinaceous toxins of bacterial, fungal, plant or animal origin, including fragments and/or variants thereof. It will also be understood by a person skilled in the relevant art that the term “prodrug” as used in this application generally refers to a precursor or derivative form of a pharmaceutically active substance that is less cytotoxic to target cells compared to the pharmaceutically active substance and is capable of being activated or converted into the more pharmaceutically active substance.

The invention also relates to a pharmaceutical composition comprising modifications of the HIV-1 gp41-binding single-chain coiled coil according to the invention as described above and a pharmaceutically acceptable carrier.

It will be understood by a person skilled in the relevant art that the compositions of the present invention, including but not limited to Alphabodies, can be formulated into pharmaceutical compositions for administration in a manner customary for administration of such materials using standard pharmaceutical formulation chemistries and methodologies. It will also be understood by a person skilled in the relevant art that such pharmaceutical compositions may include one or more excipients, carriers, stabilizers or other pharmaceutically inactive compounds, such as, but not, limited to, wetting or emulsifying agents, pH buffering substances and the like. Pharmaceutically acceptable salts can also be included therein. The pharmaceutical formulations of the invention may also comprise or be combined with other therapeutic agents.

The Alphabodies of the present invention may be administered parenterally, including, but not limited to, intramuscular, intravenous, subcutaneous or intraperitoneal injection or infusion, and via transdermal or transmucosal administration. Alternatively, administration of the Alphabodies of the present invention may be topical, including, but not limited to anal or vaginal administration. The therapeutically effective doses may vary according to body weight and the timing and duration of administration will be determined by specific clinical research protocols.

The primary usage of anti-gp41 Alphabodies is to bind to a gp41 sub-region and thereby block 6-helix bundle formation. There are different methods by which such Alphabodies can be obtained. The most well-known is to apply combinatorial libraries (e.g., a library with selected randomized amino acid positions) and a suitable display technique (e.g., phage-display) in combination with a suitable selection method (e.g., biopanning). The usage of a combinatorial library approach has been contemplated by the present applicants for the purpose of generating gp41-binding Alphabodies and for the further maturation (optimization) of initial binders.

Further embodiments of the present invention are directed to the use of the D-isomer of gp41 or a fragment thereof (contrary to the naturally occurring L-isoform) for the screening or biopanning of a natural L-isomeric Alphabody library. A D-isomeric gp41 binding L-isomeric Alphabody emerging from such a screening or biopanning can then be used to construct a D-isomeric Alphabody with the same sequence, directed against natural L-isomeric gp41. D-isomeric Alphabodies benefit in vivo from their resistance to proteolytic cleavage and potentially from altered pharmacokinetics.

In Examples 1 and 2, two alternative methods are provided which are herein fully elaborated. These methods are known in the art as design by grafting. They rely in part on the structural similarities between an Alphabody and a gp41 N-trimer. Indeed, with respect to its constitution, an Alphabody is essentially also a trimeric parallel coiled coil, although there are major differences between both types of coiled coils: (i) an Alphabody is a single-chain protein molecule whereas an N-trimer is a complex of three individual, non-covalently associated HR1 fragments; (ii) the core of an Alphabody consists of a majority of isoleucines, while the core of an N-trimer is relatively heterogeneous (4 isoleucines out of 11 heptad a/d-positions within the fragment 543 to 582 of a HXB2 strain; the other a/d-positions are occupied by leucine, glutamine and threonine); (iii) an Alphabody can exist as a parallel or antiparallel coiled coil, whereas an N-trimer is invariably parallel; (iv) an Alphabody is not composed of, or derived from, a naturally occurring amino acid sequence but it is a non-natural sequence that is optimized to adopt a stable fold. Nonetheless, in a parallel or antiparallel Alphabody there is at least one pair of parallel helices which are in theory suited for redesign with the aim of mimicking an N-trimer groove of gp41. One such method of redesign is known in the art as grafting. While grafting is a non-obvious approach because of the multitude of simultaneously “transplanted” amino acid side chains, it may form a rapid and convenient way to obtain lead constructs which may optionally be further optimized in subsequent optimization rounds.

Accordingly, applicants have contemplated the redesign of an Alphabody groove to mimic an N-trimer groove by way of grafting the N-trimer/HR2 interface residues onto the Alphabody. Similarly, applicants have also contemplated the redesign of a single Alphabody helix to mimic the HR2 surface that makes contact with an N-timer in a 6-helix bundle complex. Applicants have further contemplated the redesign of both a groove between two helices in an Alphabody and the surface of the third helix of the same Alphabody, so as to simultaneously display an N-trimer-like binding groove and an HR2-like helical surface. As will be appreciated by persons skilled in the art, such redesign-by-grafting is not at all obvious because the grafted side-chains are attached onto a non-native (non-viral, foreign) protein scaffold. There they “arrive” in a context of other side chains that are different from their native context, which may give rise to conformation-disturbing effects and loss of affinity. Further, in the case of a bifunctional Alphabody construct, there is a high risk for self-association.

On condition of a successful design of an N-trimer-mimicking binding groove in the context of an Alphabody, the latter may also be used for other applications than gp41 binding. Indeed such mimic can be used to search (screen) for additional molecules, not related to an Alphabody, that can bind the N-trimer-mimicking Alphabody and cross-react with gp41 HR2 fragments in viral spikes. Therefore, applicants have contemplated a method of identifying a chemical compound or molecule that binds a gp41 N-trimer mimicking Alphabody, and inhibits HIV infection, this method comprising (i) exposing a candidate chemical compound or molecule to said N-trimer-mimicking Alphabody, (ii) determining the binding of said candidate chemical compound or molecule for said Alphabody, (iii) selecting said candidate chemical compound or molecule if binding occurs, and (iv) assessing the HIV inhibitory activity of said selected candidate chemical compound or molecule by a suitable HIV inhibition assay.

The Alphabodies of the present invention can be synthesized using chemical synthesis methods known in the art. Alternatively, the Alphabodies of the present invention can be produced by genetic engineering techniques. Thus, the invention relates to a nucleic acid, for example, DNA or RNA, encoding an Alphabody of the present invention; an expression vector comprising said nucleic acid; a host cell transformed or infected with said nucleic acid or expression vector as well as a method for the production of an Alphabody of the invention comprising transforming or infecting a host cell with the nucleic acid according to the invention, preferably the vector according to the invention.

The Alphabodies of the present invention can be synthesized using chemical synthesis methods known in the art. Alternatively, the Alphabodies of the present invention can be produced by genetic engineering techniques. Thus, the invention relates to a nucleic acid, for example, DNA or RNA, encoding an Alphabody of the present invention.

The invention also relates to a vector, preferably an expression vector, comprising said nucleic acid encoding an Alphabody of the present invention.

As used herein, the term “vector” is used in reference to nucleic acid molecules that transfer DNA segment(s) from one cell to another.

The term “expression vector” as used herein refers to a recombinant nucleic acid molecule that contains a desired nucleic acid target sequence and appropriate nucleic acid sequences necessary for the expression of nucleic acid or amino acid sequences in a host. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The invention further relates to a host cell transformed or infected with said nucleic acid, vector or expression vector.

As used herein, the term “host” or “host cell” refers to any eukaryotic or prokaryotic cell (e.g. bacterial cells such as E. coli, yeast cells such as P. pastoris, mammalian cells, avian cells, amphibian cells, plant cells, fish cells, and insect cells), whether located in vitro or in vivo. For example, host cells may be located in a transgenic animal.

It may be understood by a person skilled in the relevant art that Alphabodies of the present invention can be made by recombinant DNA methods. DNA encoding the Alphabodies of the invention can be readily synthesized using conventional procedures. Once prepared, the DNA can be placed into expression vectors, which are then transformed or transfected into host cells such as E. coli or P. pastoris, in order to obtain the synthesis of Alphabodies in the recombinant host cells.

Thus, the invention also relates to a method for the production of an Alphabody of the invention comprising transforming, transfecting or infecting a host cell with the nucleic acid according to the invention, preferably the vector according to the invention, more preferably an expression vector according to the invention.

The terms “transformation” and “transfection” as used herein refer to the introduction of foreign DNA into respectively prokaryotic and eukaryotic cells. These procedures may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

Further, the invention relates to the usage of N-trimer-mimicking Alphabodies for usage as a drug-screening tool in a competition assay. The latter usage corresponds to a method of identifying a chemical compound or molecule that inhibits HIV infection, comprising (i) exposing Env-expressing cells simultaneously to an N-trimer-mimicking Alphabody and to a candidate chemical compound or molecule, (ii) determining competitive binding of said candidate chemical compound or molecule with respect to said N-trimer-mimicking Alphabody, (iii) selecting said candidate chemical compound or molecule if competitive binding occurs, and (iv) assessing the HIV inhibitory activity of said selected candidate chemical compound or molecule by a suitable HIV inhibition assay.

Further, the invention relates to the usage of gp41-mimicking Alphabodies as a vaccine. The latter usage corresponds to a method of eliciting an immune response to HIV in an individual, comprising exposing (or immunizing) said individual to a gp41-mimicking Alphabody.

### EXAMPLES

### Example 1

### N-Trimer Groove-Grafted Alphabodies

The aim of the present example is to demonstrate a practically feasible method to generate a gp41 N-trimer-mimicking Alphabody.

Applicants have analyzed the crystallographically determined structure of the 5-helix bundle in complex with the Fab antigen-binding domain D5 (Root et al. Science 2001, 291:884-888; PDB structure 2CMR). FIG. 7, panel A, shows a schematic representation of the N-trimer part of the 5-helix bundle.

According to the authors, the N-trimer groove residues that constitute the interface with HR2 are located at heptad e- and g-positions. However, according to our own structural analysis, at least the b- and c-residues should be taken into account as well. Therefore, when grafting gp41 groove residues onto structurally equivalent positions in an Alphabody, the set amino acid residues located at b-, c-, e- and g-positions are to be considered, unlike what is suggested in FIG. 4 of Root et al. (ibid), where only the e- and g-residues are depicted as interface residues.

In the legend to FIG. 7, some structural aspects relating to the grafting of specific amino acid residues from the N-trimer groove onto an Alphabody are explained.

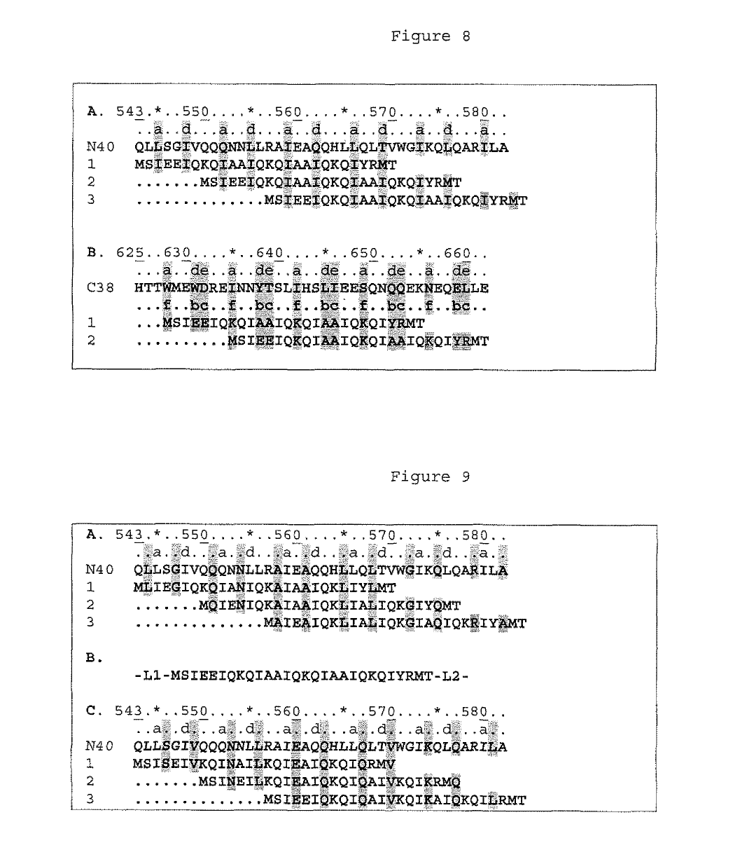

In FIG. 8A, sequence alignments of Alphabody denoted “scAB013” with HR1 sequence denoted “N-40” are provided in three different frames. scAB013 is a specific Alphabody that has been selected by the present applicants because of its high thermostability. scAB013 is defined in terms of its amino acid sequence as SEQ ID No: 2. In structural terms, the first Alphabody helix (“helix A”, “heptad repeat sequence 1”) is connected to the second helix (“helix B”, “heptad repeat sequence 2”) by a linker sequence (“L1”) and the second Alphabody helix is connected to the third helix (“helix C”, “heptad repeat sequence 3”) by a linker sequence (“L2”). This means that, irrespective of the orientation of helix B with respect to the mutually parallel helices A and C (thus irrespective of whether the Alphabody is parallel or antiparallel), helices A and C form a pair of parallel helices that are similar in structure and orientation to any pair of helices in an N-trimer.

The alignments in FIG. 8A form the basis of the grafting procedure. In view of the structural similarity between Alphabody and N-trimer grooves, N-trimer positions c and g are to be grafted on an Alphabody A-helix and positions b and a are to be grafted on the C-helix. This gives rise to the initial (non-optimized) Alphabody sequences with grafted groove residues, as depicted in FIG. 9.

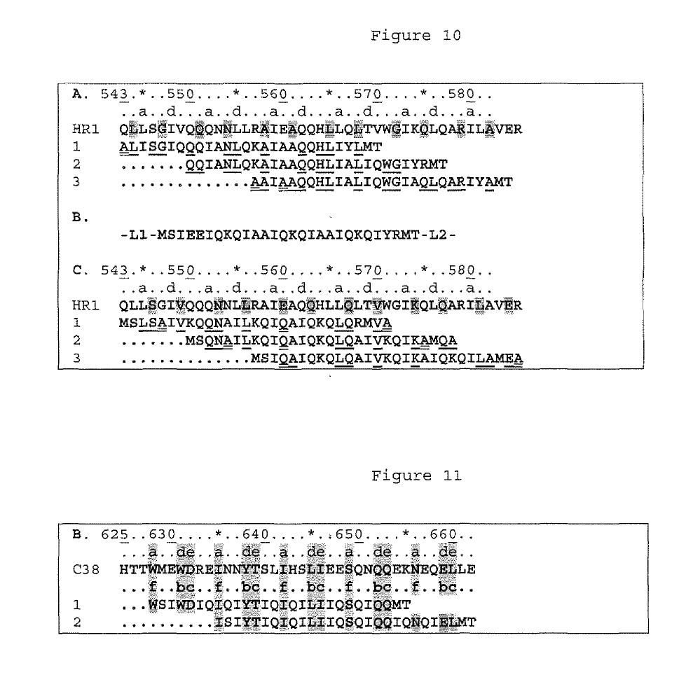

As will be appreciated by persons skilled in the art, straightforward “copy-pasting” of interface residues from one structure onto another will usually not lead to a full transfer of functionality; in other words, binding affinity will usually be lost or at least significantly diminished. Therefore, all amino acid residues that were grafted on a sequence basis as shown in FIG. 9 were effectively placed on a 3-D model of the scAB013 Alphabody by mutating the latter with standard torsion angles. Next, each mutated residue was examined in its structural context. In case this analysis casted doubt on the structural compatibility, then alternative substitutions were considered. The latter are shown in FIG. 10 as double-underlined residues. As is seen there, most of the uncertain residues were mutated into alanines which were considered generally safer.

The final, structurally optimized Alphabody constructs with grafted N-trimer-like binding grooves are shown in FIG. 13. The linker sequences selected to connect helices A to B (L1) and helices B to C (L2) were chosen to be each having the 6-residue amino acid sequence “glycine-glycine-serine-serine-glycine-glycine”. The combined sequences are provided as SEQ ID No: 3 (denoted “scAB013_N1”), SEQ ID No: 4 (“scAB013_N2”) and SEQ ID No: 5 (“scAB013_N3”).

### Example 2

### HR2 Binding Site-Grafted Alphabodies

The aim of the present example is to demonstrate a practically feasible method to generate an Alphabody that mimics the HR2 surface that makes contact with an N-trimer groove in a 6-helix bundle (HR2 binding site-grafted or HR2-mimicking Alphabody). Essentially the same strategy was followed as in EXAMPLE 1, with specific modifications as discussed hereinafter.

FIG. 7, panel A, lower helical wheel, shows a schematic representation of an HR2 helix of the 5-helix bundle.

According to the authors, the HR2 residues that constitute the interface with an N-trimer are located at heptad a- and d-positions. However, according to our own structural analysis, at least the e-residues should be taken into account as well. Therefore, when grafting gp41 groove residues onto structurally equivalent positions in an Alphabody, the set amino acid residues located at a-, d- and e-positions are to be considered, unlike what is suggested in FIG. 4 of Root et al. (ibid), where only the a- and d-residues are depicted as interface residues.

In the legend to FIG. 7, some structural aspects relating to the grafting of specific amino acid residues from the HR2 helix onto an Alphabody are explained.

In FIG. 8B, sequence alignments of Alphabody scAB013 (SEQ ID No: 2) with HR2 sequence denoted “C-38” are provided in two different frames. Special in this case is that heptad positions a, d and e in HR2 are to be grafted on maximally exposed positions in an Alphabody so as to be fully accessible for gp41 N-trimer binding. The Alphabody positions chosen for this purpose are b-, c- and f-positions, with the mapping as explained in the legend to FIG. 7. This mapping was used in the alignments shown in FIG. 8B.

With respect to the type of Alphabody, it does not make an essential difference whether the latter is parallel or antiparallel, because the only aim is to make the Alphabody bind to a gp41 N-trimer groove through a single alpha-helix. Which helix (A, B or C) is chosen is in principle also not relevant, but in view of the ultimate aim to develop bifunctional Alphabodies, the most optimal choice is the B-helix.

The alignments in FIG. 8B form the basis of the grafting procedure. There, all HR2 a-residues are transferred to Alphabody f-positions, and HR2 d- and e-residues are transferred to Alphabody b- and c-positions, respectively. This gives rise to the initial (non-optimized) Alphabody sequences with grafted HR2 residues, as depicted in FIG. 11.

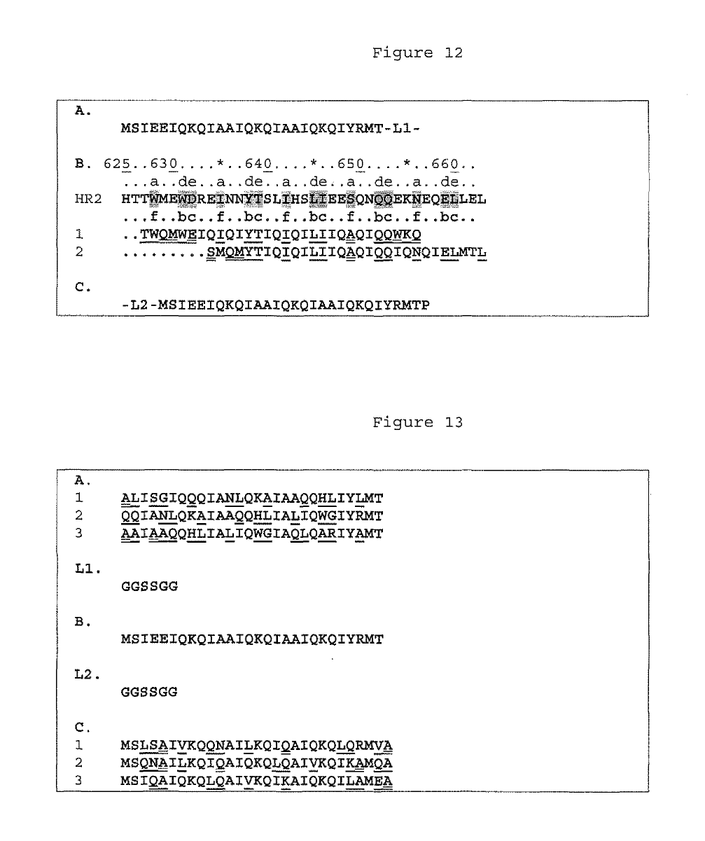

As in EXAMPLE 1, all amino acid residues that were grafted on a sequence basis as shown in FIG. 11 were effectively placed on a 3-D model of the scAB013 Alphabody by mutating the latter with standard torsion angles. Next, each mutated residue was examined in its structural context. In case this analysis casted doubt on the structural compatibility, then alternative substitutions were considered. The latter are shown in FIG. 12 as double-underlined residues. Unlike in EXAMPLE 1, most uncertain residues were this time not mutated into alanines, but into isosteric or slightly larger residue types to compensate for the helical bending which is towards the center of an Alphabody, whereas the bending should be opposite for ideal binding to a gp41 N-trimer.

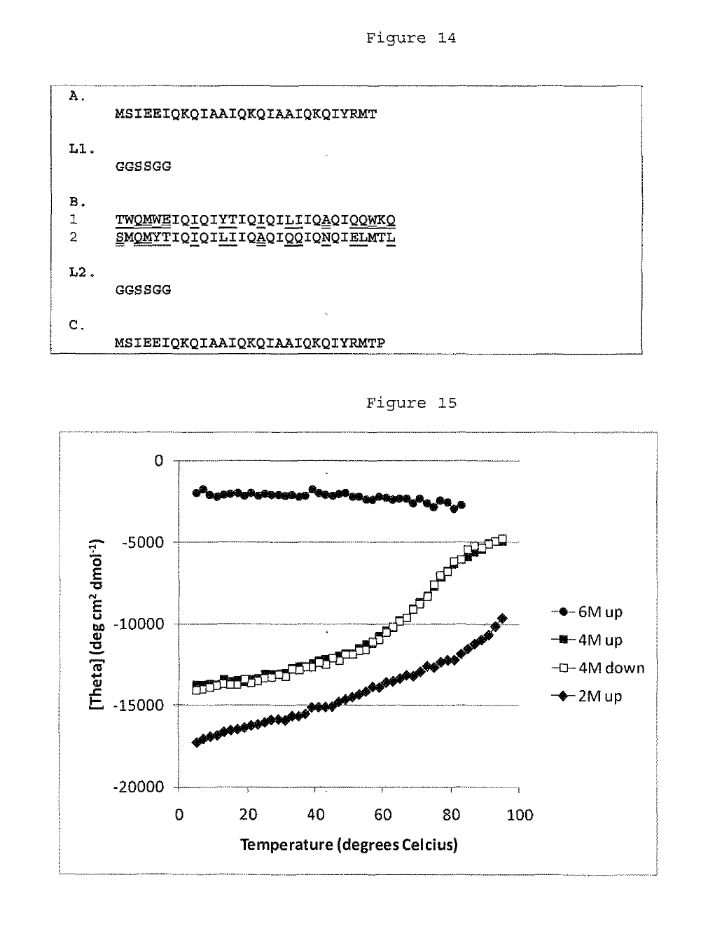

The final, structurally optimized Alphabody constructs with a grafted HR2-like surface are shown in FIG. 14. The linker sequences selected to connect the Alphabody helices were again chosen to have the 6-residue amino acid sequence “glycine-glycine-serine-serine-glycine-glycine”. The combined sequences are provided as SEQ ID No: 6 (denoted “scAB013_C1”) and SEQ ID No: 7 (“scAB013_C2”).

### Example 3

### Soluble Expression and Characterization of scAB013_N3

The aim of the present example is to demonstrate that a gp41 N-trimer-mimicking Alphabody can be obtained by recombinant expression in E. coli, purified from the cytoplasmic fraction, and physico-chemically characterized. A second aim is to demonstrate that the obtained Alphabody binds in vitro to its cognate target sequence.

A synthetic gene for scAB013_N3, N-terminally appended with a (His)6 tag, was purchased (GeneArt). This coding sequence was subcloned into the pET16b vector (Novagen). The resulting construct was transformed into the host E. coli strain BL21(DE3) harboring a chromosomal copy of the T7 polymerase gene under control of the lacUV5 promoter (DE3 lysogen). Transformed cells were grown in medium supplemented with ampicillin and protein expression was induced by the addition of IPTG to exponentially growing cultures. Cells containing the expressed Alphabodies were collected by centrifugation and the pellets were resuspended in 50 mM Tris, 500 mM NaCl, pH 7.8. Cells were then disrupted by sonication and spun down for cell debris removal. The cleared supernatants were applied onto a HITrap IMAC HP column (GE Healthcare) loaded with Ni²⁺ ions. Bound proteins were eluted by applying an imidazole gradient from 5 to 1000 mM. Alphabody-containing fractions were pooled, concentrated and loaded on a Superdex 75 size exclusion chromatography (SEC) column (GE Healthcare). During this final purification step, the buffer was changed to 50 mM Iris, 150 mM NaCl, pH 7.8.

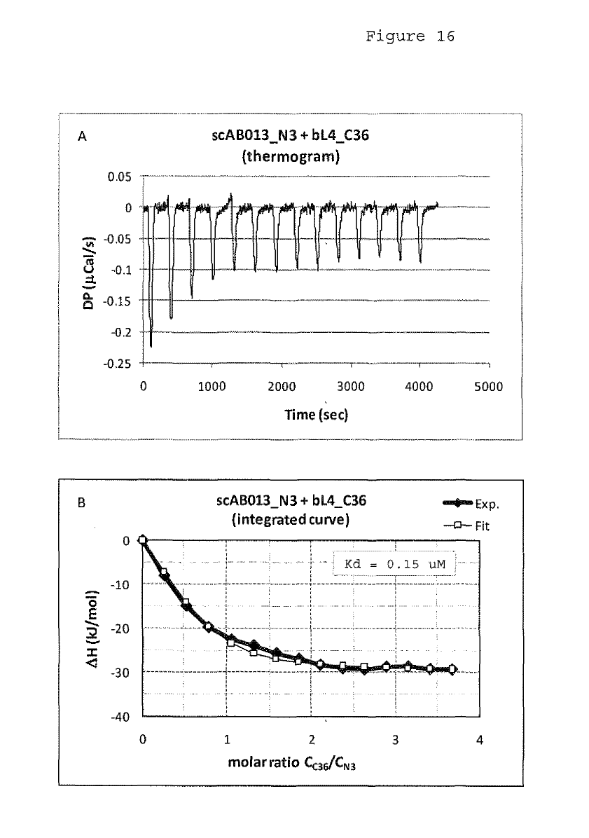

FIG. 15 shows CD thermoscans at 222 nM for the purified scAB013_N3 Alphabody in 20 mM PBS, 150 mM NaCl, pH 7.2 and in varying concentrations of GuHCl. Three thermoscans were performed from low to high temperature (“up scans”) in the presence of 2 M, 4 M and 6 M GuHCl, respectively (filled symbols). One high to low temperature scan (“down scan”; open symbols) was recorded as well. The 2 M up scan shows that the scAB013_N3 construct remains quantitatively folded over almost the entire temperature range. The start of thermal unfolding is observed at temperatures exceeding 90° C. (curve-fitted Tm=101° C.). The 4 M up scan shows nearly complete thermal unfolding with a fitted Tm=74° C. The 4 M down scan shows that the thermal unfolding process is fully reversible (open squares coincide with filled squares). The 6 M upscan is indicative of unfolded protein over the full temperature range. Taken together, the thermal unfolding experiments show that the scAB013_N3 Alphabody is highly thermostable and that the folding/unfolding process is fully reversible.

FIGS. 16A and 16B show the results of an isothermal titration calorimetry (ITC) experiment on scAB013_N3 titrated with biotinylated C36 peptide. The biotinylated C36 peptide, referred to as “bL4_C36”, corresponds to the cognate target sequence of the Alphabody. It consists of residues 628 to 663 of the HIV-1 HXB2 Env sequence (SEQ ID No: 1), the latter being N-terminally biotinylated and C-terminally amidated and wherein the biotin group is attached to the C36 sequence through a 4-residue Gly/Ser linker (-Gly-Ser-Gly-Ser-). The thermogram (FIG. 16A) shows small exothermic heat releases upon addition of bL4_C36 which gradually decrease up to the point of saturation near a molar ratio of 1. The baseline-corrected and integrated plot (FIG. 16B) was curve fitted in accordance with a 1:1 binding model. This yielded the following thermodynamic parameters: ΔH=−30 kJ/mol and Kd=150 nM. Taken together, the ITC experiments show that the scAB013_N3 Alphabody binds to its cognate target sequence with a reasonably high affinity. The latter is not evident in view of the fact that the construct was based on a rational design involving 25 substitutions (underlined residues in FIG. 13) compared to the original scAB013 Alphabody.

1. An HIV-1 gp41-binding single-chain coiled coil having a single contiguous amino acid chain with the formula HRS1-L1-HRS2-L2-HRS3, optionally supplemented with N- and C-terminal extensions resulting in the formula N-HRS1-L1-HRS2-L2-HRS3-C, wherein
a) each of HRS1, HRS2 and HRS3 is independently a heptad repeat sequence consisting of 2 to 7 consecutive heptad repeat units, at least 50% of all heptad a- and d-positions are occupied by isoleucine residues, and HRS1, HRS2 and HRS3 together constitute a 3-stranded alpha-helical coiled-coil structure;
b) each of L1 and L2 are independently a linker fragment, covalently connecting HRS1 to HRS2 and HRS2 to HRS3, respectively, starting and ending with a proline or glycine, and consisting of 3 to 30 amino acid residues of which at least 50% are selected from the group proline, glycine, serine;
c) N and C are independently an optional extension, covalently connected to the N- and C-terminal end of HRS1 and HRS3, respectively, this connection being marked by a helix-breaking proline or glycine.

2. The single-chain coiled coil of claim 1 which binds to the extracellular domain of HIV-1 gp41, said extracellular domain being defined as amino acid residues 1 to 683 of SEQ ID NO: 1.

3. The single-chain coiled coil of claim 1 which binds to the HR1 fragment of HIV-1 gp41, said HR1 fragment being defined as amino acid residues 546 to 581 of SEQ ID NO: 1, or which binds to the HR2 fragment of HIV-1 gp41, said HR2 fragment being defined as amino acid residues 628 to 661 of SEQ ID NO: 1.

4. (canceled)

5. The single-chain coiled coil of claim 1 which binds simultaneously to the HR1 and HR2 fragments of HIV-1 gp41, said HR1 and HR2 fragments being defined as amino acid residues 546 to 581 and 628 to 661 of SEQ ID NO: 1, respectively.

6. The single-chain coiled coil of claim 1,
wherein the binding to HIV-1 gp41 is characterized by a dissociation constant (Kd) or half maximal effective concentration (EC50) in the submicromolar range.

7. The single-chain coiled coil of claim 1,
wherein the binding to HIV-1 gp41 inhibits HIV env-mediated cell-cell fusion, characterized by a half maximal inhibitory concentration (IC50) in the submicromolar range, or
wherein the binding to HIV-1 gp41 inhibits HIV viral entry, characterized by a half maximal inhibitory concentration (IC50) in the submicromolar range, or
wherein the binding to HIV-1 gp41 inhibits HIV viral replication, characterized by a half maximal inhibitory concentration (IC50) in the submicromolar range.

8. (canceled)

9. (canceled)

10. The single-chain coiled coil of claim 1, wherein the binding to HIV-1 gp41 inhibits HIV infection of mammalian cells.

11. The single-chain coiled coil of claim 10 wherein the binding to HIV-1 gp41 inhibits HIV infection of human cells.

12. A method of identifying a chemical compound or molecule that binds a single-chain coiled coil according to claim 1 and inhibits HIV infection, comprising exposing a candidate chemical compound or molecule to said single-chain coiled coil, determining the binding of said candidate chemical compound or molecule for said single-chain coiled coil, selecting said candidate chemical compound or molecule if binding occurs, and assessing the HIV inhibitory activity of said selected candidate chemical compound or molecule.

13. A method of identifying a chemical compound or molecule that inhibits HIV infection, comprising exposing env-expressing cells simultaneously to a single-chain coiled coil according to claim 1 and to a candidate chemical compound or molecule, determining competitive binding of said candidate chemical compound or molecule with respect to said single-chain coiled coil, selecting said candidate chemical compound or molecule if competitive binding occurs, and assessing the HIV inhibitory activity of said selected candidate chemical compound or molecule.

14. A pharmaceutical composition comprising an HIV-1 gp41-binding single-chain coiled coil according to claim 1 and a pharmaceutically acceptable carrier.

15. A method of inhibiting HIV infection in an individual, comprising administering to the individual the HIV-1 gp41-binding single-chain coiled coil according to claim 1.

16. A method of eliciting an immune response to HIV in an individual, comprising exposing said individual to the single chain coiled coil according to claim 1 for use in eliciting an immune response to HIV in an individual.

17. A method of vaccinating against HIV infection in an individual, comprising administering to the individual the HIV-1 gp41-binding single-chain coiled coil according to claim 1.

18. A method of treating or preventing HIV infection in an individual, comprising administering to the individual the HIV-1 gp41-binding single-chain coiled coil according to claim 1.

19. A method of diagnosing HIV infection in an individual, comprising administering to the individual an HIV-1 gp41-binding single-chain coiled coil according to claim 1.

20. A nucleic acid molecule encoding an amino acid sequence of an HIV-1 gp41-binding single-chain coiled coil according to claim 1.

21. A host cell comprising a nucleic acid molecule according to claim 20.
